(19) United States
(12) Patent Application Publication
(10) Pub. No.: US 2025/0149187 A1
(43) Pub. Date: May 8, 2025

(54) FACT-AWARE SYNOPTIC REPORT GENERATION USING INSTRUCTION-TUNED LANGUAGE MODELS

(71) Applicant: Siemens Healthineers AG, Forchheim (DE)

(72) Inventors: Rikhiya Ghosh, Livingston, NJ (US); Sanjeev Kumar Karn, Pennington, NJ (US); Poikavila Ullaskrishnan, Lebanon, NH (US); Oladimeji Farri, Upper Saddle River, NJ (US); Dorin Comaniciu, Princeton, NJ (US)

(21) Appl. No.: 18/897,178

(22) Filed: Sep. 26, 2024

Related U.S. Application Data
(60) Provisional application No. 63/596,695, filed on Nov. 7, 2023.

(51) Int. Cl. G16H 70/20; G06F 40/279; G06F 40/40; G16H 30/40

(52) U.S. Cl. CPC G16H 70/20; G06F 40/279; G06F 40/40; G16H 30/40

(57) ABSTRACT

Systems and methods for generating radiology passages are provided. An input common data element and one or more associated input values are received. A radiology passage is generated based on the input common data element and the one or more associated input values using a trained language model. The generated radiology passage is output.

### CROSS-REFERENCE TO RELATED APPLICATIONS

This application claims the benefit of U.S. Provisional Application No. 63/596,695, filed Nov. 7, 2023, the disclosure of which is herein incorporated by reference in its entirety.

### TECHNICAL FIELD

The present invention relates generally to AI/ML (artificial intelligence/machine learning) based systems for performing medical analysis tasks, and in particular to fact-aware synoptic report generation using instruction-tuned language models.

### BACKGROUND

Traditional radiology reporting involves a radiologist writing free text to describe imaging observations and their differential diagnosis from radiographic medical images of patients. However, such free text writing has resulted in inconsistencies, information gaps, variable reporting styles, and difficulty in automatically extracting information for continuity of care, integration of medical records, patient understanding, and legal and medical documentation. This has resulted in standardized structured radiology reporting.

Synoptic report generation refers to the automatic restructuring of radiological data from free text reports to a synoptic format for standardizing radiology lexicon and ontologies. Synoptic report generation aids the radiologist in generating reports while avoiding missing information and ensuring a seamless information transfer from radiology reporting to other clinicians and clinical databases. However, there is no standard radiology reporting structure that has been universally agreed upon.

### BRIEF SUMMARY OF THE INVENTION

In accordance with one or more embodiments, systems and methods for generating radiology passages are provided. An input common data element and one or more associated input values are received. A radiology passage is generated based on the input common data element and the one or more associated input values using a trained language model. The generated radiology passage is output.

In one embodiment, the trained language model is trained by receiving text-based radiological data comprising one or more passages. A concept is extracted from each of the one or more passages. For each respective passage of the one or more passages, the concept extracted from the respective passage is mapped to a common data element and one or more associated values, thereby resulting in pairs of 1) the respective passage and 2) the common data element and the one or more associated values. The language model is trained for generating the radiology passage based on the pairs of 1) the passages and 2) the common data elements and the one or more associated values.

In one embodiment, the language model is fine-tuned via instruction tuning based on the pairs of 1) the passages and 2) the common data elements and the one or more associated values comprises. In one embodiment, the language model is trained via reinforcement learning by evaluating the common data elements against passaged generated by the language model.

In one embodiment, in response to determining that the common data element is not available for mapping, the concept extracted from the respective passage is mapped to an entity of an ontology. A ranking of entities is determined for the ontology from each of a plurality of entity linking models based on the concept extracted from the respective passage. The entity of the ontology is determined based on the ranking of the entities.

In one embodiment, each of the one or more passages is encoded into features using a machine learning based encoder network. The concept is classified to a common data element and the one or more associated values using a machine learning based classifier model.

In accordance with one or more embodiments, systems and method for training a language model for generating radiology passages are provided. Text-based radiological data comprising one or more passages is received. A concept is extracted from each of the one or more passages. For each respective passage of the one or more passages, the concept extracted from the respective passage is mapped to a common data element and one or more associated values, thereby resulting in pairs of 1) the respective passage and 2) the common data element and the one or more associated values. A language model is trained for generating a radiology passage based on the pairs of 1) the passages and 2) the common data elements and the one or more associated values. The trained language model is output.

These and other advantages of the invention will be apparent to those of ordinary skill in the art by reference to the following detailed description and the accompanying drawings.

### BRIEF DESCRIPTION OF THE DRAWINGS

FIG. 1 shows a method for training a language model for generating passages, in accordance with one or more embodiments;

FIG. 2 shows a workflow for training a language model for generating passages, in accordance with one or more embodiments;

FIG. 3 shows a workflow for mapping concepts extracted from passages to entities of ontologies, in accordance with one or more embodiments;

FIG. 4 shows exemplary instructions for instruction tuning, in accordance with one or more embodiments;

FIG. 5 shows a workflow for training and applying a PLM for mapping passages to common data elements, in accordance with one or more embodiments;

FIG. 6 shows an interface of an annotation tool for annotating radiological data with common data elements and associated values, in accordance with one or more embodiments;

FIG. 7 shows a method 700 for generating radiological passages using a language model, in accordance with one or more embodiments;

FIG. 8 shows an exemplary artificial neural network that may be used to implement one or more embodiments;

FIG. 9 shows a convolutional neural network that may be used to implement one or more embodiments;

FIG. 10 shows a schematic structure of a recurrent machine learning model that may be used to implement one or more embodiments; and

FIG. 11 shows a high-level block diagram of a computer that may be used to implement one or more embodiments.

### DETAILED DESCRIPTION

The present invention generally relates to methods and systems for fact-aware synoptic report generation using instruction-tuned language models. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Embodiments described herein provide for synoptic report generation using an instruction-tuned language model. The language model is fine-tuned via instruction tuning according to a two-phase approach. During a synoptic mapper phase, medical concepts extracted from passages of text-based radiologic data are mapped to common data elements, thereby resulting in pairs of passages and common data elements. During, a synoptic report generator phase, a language model is fine-tuned via instruction tuning and reinforcement learning using the pairs of passages and common data elements. Advantageously, the language model generates a standard structured report that is readable for clinicians and patients while avoiding missing information.

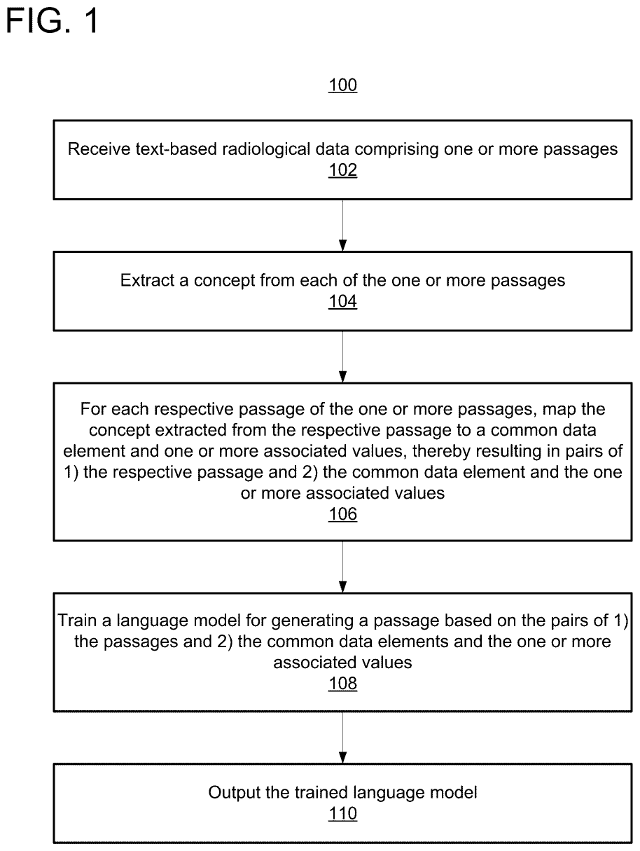

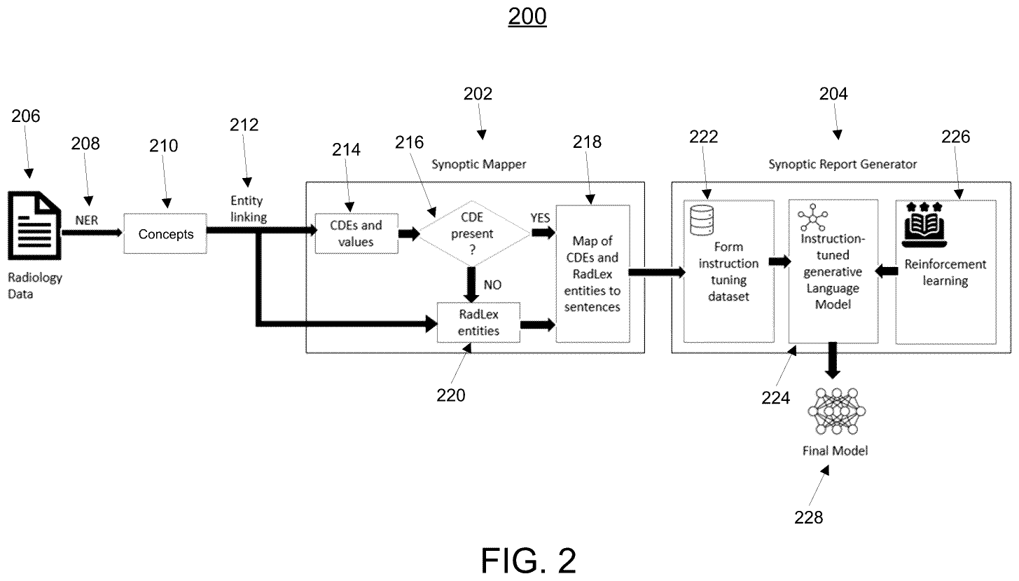

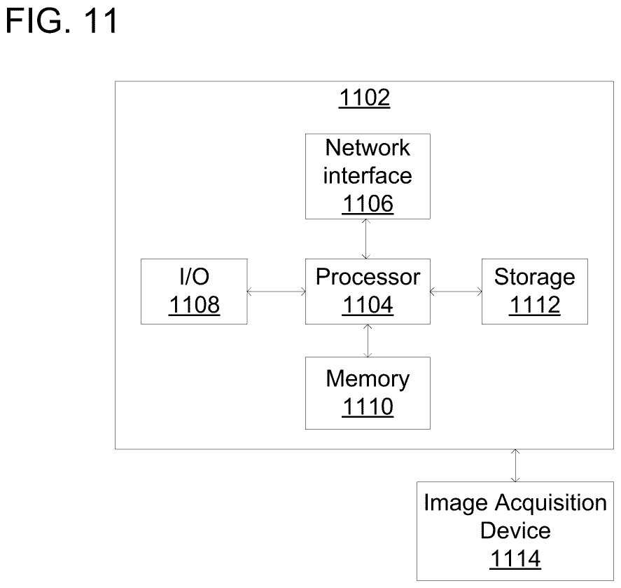

FIG. 1 shows a method 100 for training a language model for generating passages, in accordance with one or more embodiments. The steps and sub-steps of method 100 may be performed by one or more suitable computing devices, such as, e.g., computer 1102 of FIG. 11. FIG. 2 shows a workflow 200 for training a language model for generating passages, in accordance with one or more embodiments. Workflow 200 comprises two phases: a synoptic mapper phase 202 and a synoptic report generator phase 204. Synoptic mapper phase 202 corresponds to steps 102-106 of FIG. 1. Synoptic report generator phase 204 corresponds to step 108 of FIG. 1. FIG. 1 and FIG. 2 will be described together. Method 100 of FIG. 1 and workflow 200 of FIG. 2 are performed during an offline or training stage for training the language model.

At step 102 of FIG. 1, text-based radiological data comprising one or more passages is received. In one example, as shown in workflow 200 of FIG. 2, the text-based radiological data is radiology data 206.

The text-based radiological data may comprise any data related to medical imaging that is based on text. The text-based radiological data may be in natural language, free text, or any other suitable format. In one example, the text-based radiological data comprises radiology reports or portions thereof, such as, e.g., the findings section of a radiology report. The text-based radiological data comprises one or more passages. The one or more passages may include, for example, a phrase, a sentence, a paragraph, a section, or any other portion of the text-based radiological data.

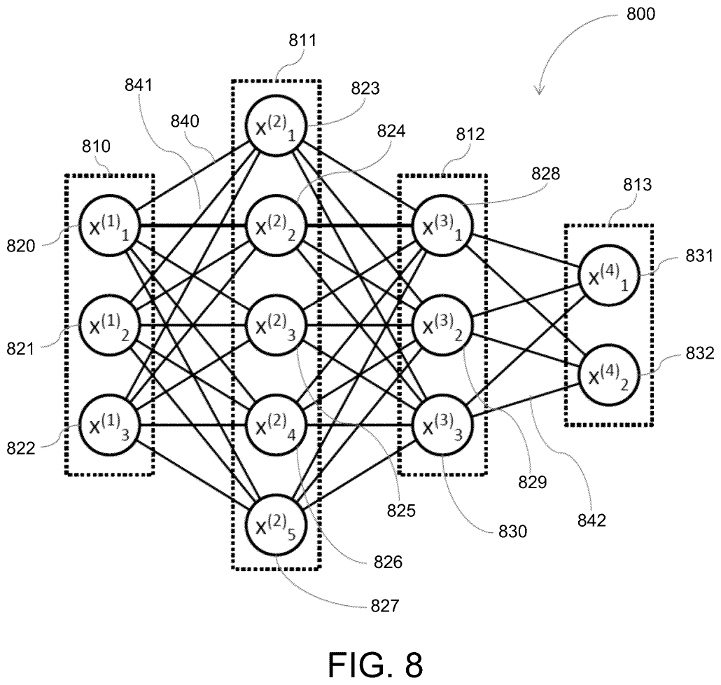

The text-based radiological data may be received, for example, by directly receiving the text-based radiological data from a user via an input/output (I/O) device (e.g., I/O 808 of FIG. 8), by loading the text-based radiological data from a storage or memory of a computer system (e.g., storage 812 or memory 810 of computer 802 of FIG. 8), or by receiving the text-based radiological data from a remote computer system (e.g., computer 802 of FIG. 8). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, a concept is extracted from each of the one or more passages. In one example, as shown in workflow 200 of FIG. 2, concepts 210 are extracted from radiology data 206 using a PLM (pretrained language model) based NER (named entity recognition) tool 208.

The concepts may be represented in any suitable form. In one embodiment, the concepts may be represented as compact, fixed-size features or embeddings (e.g., vectors) characterizing the semantic understanding of the passage. Such features may be extracted from a passage using any suitable machine learning based encoder network. For example, in one embodiment, the machine learning based encoder network is an encoder transformer model. The machine learning based encoder network receives as input each of the one or more passages and generates as output respective features representing the concept.

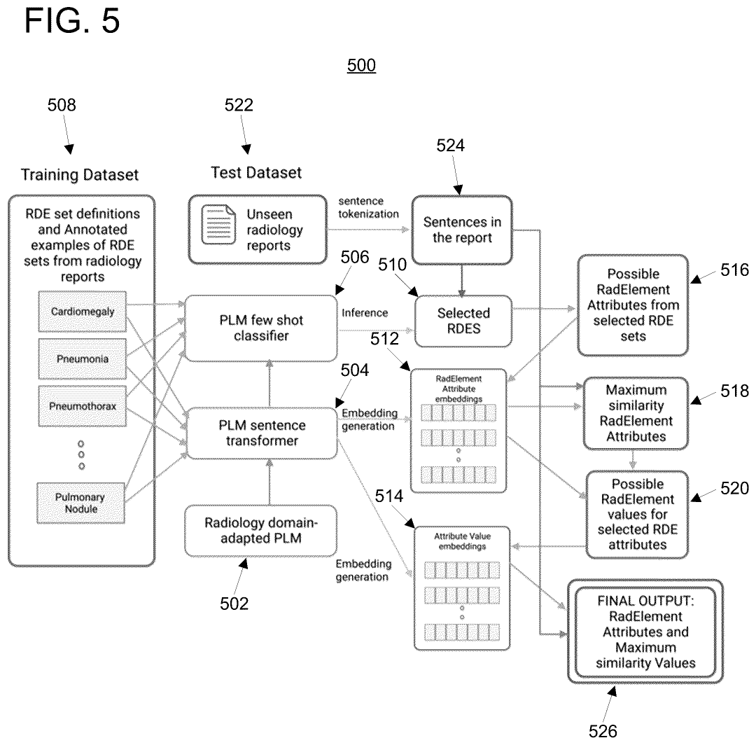

In one embodiment, a radiology domain-adapted PLM comprising a radiology domain-adapted encoder transformer model and a few-shot classifier is utilized. The PLM is trained during a prior offline or training stage, as described below in connection with FIG. 5. The encoder transformer model of the PLM may be applied at step 104 as the machine learning based encoder network to encode each passage into features. In one example, the encoder transformer model is RadLing, which uses domain-specific vocabulary and radiology ontologies (e.g., RadLex) to perform radiology report-specific intelligent masking to mask anatomies and observations alternatively to enhance understanding of the domain.

At step 106 of FIG. 1, for each respective passage of the one or more passages, the concept extracted from the respective passage is mapped to a common data element and one or more associated values, thereby resulting in pairs of 1) the respective passage and 2) the common data element and the one or more associated values. The one or more associated values are values associated with the common data element. In one example, as shown in workflow 200 of FIG. 2, concepts 210 are mapped to CDEs (common data elements) and associated values 214 by entity linking 212 and, if it is determined that CDEs are present in RAD Element (research and development common data elements) at decision block 216, the CDEs are mapped to passages (e.g., sentences) at mapping 218.

As used herein, common data elements refer to standardized, predefined properties and are associated with values. Examples of such properties of common data elements include anatomic location, shape, image number, image coordinates, dimensions, etc. In one example, the common data elements may comprise pneumothorax presence, pneumothorax lung tissue collapse, pneumothorax side, pneumothorax size and the associated values may be present, complete, left, and large respectively. The common data elements may have a standard syntax and format, and the one or more associated values may be categorically or numerically defined. Common data elements may be grouped in sets for certain abnormalities, observations, or sequence of observations. The common data elements sets may further be grouped into specialties, for example, corresponding to types of imaging, anatomies, organ systems, or types of patients.

The concepts may be mapped to the common data elements using any suitable approach. In one embodiment, a machine learning based classifier model is applied for classifying the passage to a common data element based on the extracted concept. The machine learning based classifier model may be implemented using any suitable approach. The machine learning based classifier model receives as input the extracted concept (e.g., features) and generates as output a classification to one or more common data elements.

In one embodiment the machine learning based classifier model is the few-shot classifier of the PLM. The few-shot classifier may use not only the features extracted from that particular passage, but also features from surrounding passages with higher weights to the features extracted from the particular passage.

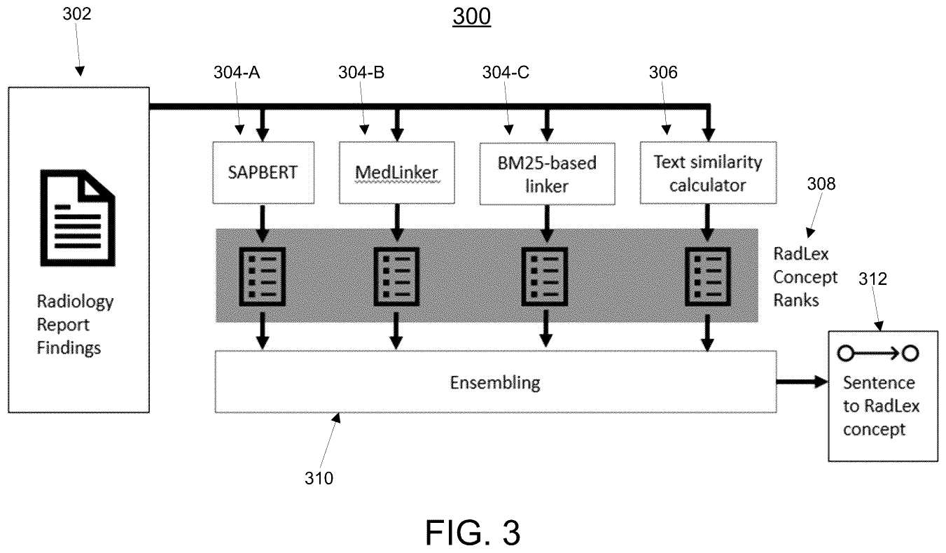

In one embodiment, if the common data elements are not available for mapping (for example, there is no corresponding common data element in RAD Elements (or any other standard that is being used)), the extracted concepts may be mapped to entities in existing ontologies to standardize the extracted concepts. For example, if there is no corresponding common data element for COPD (chronic obstructive pulmonary disease) in RAD Elements, the extracted concept may be mapped to entities of RadLex or SNOMED CT (systemized nomenclature of medicine-clinical terms). In one example, as shown in workflow 200 of FIG. 2, where the CDEs are not present in RAD Elements at decision block 216, concepts 210 are mapped to RadLex entities 220. The extracted concepts may be mapped to entities of existing ontologies using an ensemble model as described in connection with FIG. 3 below.

FIG. 3 shows a workflow 300 for mapping concepts extracted from passages to entities of ontologies, in accordance with one or more embodiments. Workflow 300 combines the understanding of a plurality of different existing (e.g., well-known) machine learning based entity linking models. The existing entity linking models in workflow 300 are, for example, SapBERT (self-alignment pre-training bidirectional encoder representations from transformers) 304-A, MedLinker 304-B, and BM25 (best matching 25)-based linker 304-C, forming an ensemble model. Any other suitable entity linking model may be utilized. Text similarity calculator 306 is also utilized in workflow 300. SapBERT 304-A, MedLinker 304-B, BM25-based linker 304-C, and text similarity calculator 306 each receives as input concepts extracted from passages (e.g., features) and definitions of the various radiology concepts from RadLex (or any other ontology) and generates as output ranks 308 of entities for RadLex (or the relevant ontology) that show the highest match between their description and the radiology passages. The ranks 308 are ensembled 310, for example, using a voting approach, to generate a final mapping 312 of the sentence (or any other passage) to an entity of RadLex.

At step 108 of FIG. 1, a language model is trained for generating a passage based on the pairs of 1) the passages and 2) the common data elements and the one or more associated values. In one embodiment, as shown in workflow 200 of FIG. 2, the mapping 218 of CDEs and RadLex entities mapped to sentences form instruction tuning dataset 222 and a pretrained generate language model 224 is trained with instruction tuning based on instruction tuning dataset 222, as well as reinforcement learning 226, to provide for final model 228.

In one embodiment, the language model is an LLM (large language model). The LLM may be any suitable pretrained deep learning based LLM. For example, the LLM may be based on the transformer architecture, which uses an attention mechanism to capture long-range dependencies in text. One example of a transformer-based architecture is GPT (generative pre-training transformer), which has a multilayer transformer decoder architecture that may be pretrained to optimize the next token prediction task. The LLM may be a radiology domain-adapted LLM, such as, e.g., Radiology GPT, RAD-Bloomz (which is a variant of BLOOM (BigScience Large Open-science Open-access Multilingual Language Model)), or Radiology LLAMA (large language model Meta AI). However, the language model may be any other suitable language model. For example, the language model may be a small language model, which uses a relatively smaller neural network, has fewer parameters, and is trained on less training data as compared with an LLM.

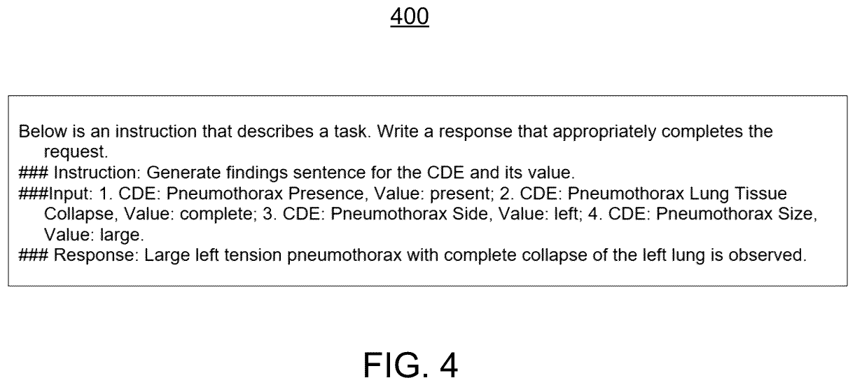

The language model may be fine-tuned via instruction tuning for generation of passages such as, e.g., radiological findings and impressions. The instruction tuning is performed using an instruction tuning dataset comprising the pairs of the passages and the common data elements and associated values. The common data elements and associated values would define the input and the passages would define the expected output. An exemplary instruction 400 for instruction tuning is provided in FIG. 4, in accordance with one or more embodiments.

In one embodiment, to account for hallucinations, the language model may be trained with reinforcement learning using the synoptic mapper to evaluate the factuality of the generated passage. The precision and recall of passages generated by the language model are evaluated against the common data elements and RadLex entities (i.e., the common data elements and the entities of the existing ontologies determined at step 106 of FIG. 1 or the CDEs and associated values 214 and RadLex entities 220 of FIG. 2). The evaluation is used as the policy for the reinforcement learning system. The reinforcement learning may be performed according to any suitable reinforcement learning algorithm, such as, e.g., PPO (proximal policy optimization) or DPO (direct policy optimization). Feedback is collected from expert radiologists using the system about whether the extracted data elements are correct or not, and the model has a positive reward for all the times the model is correct and negative reward for the incorrect answers.

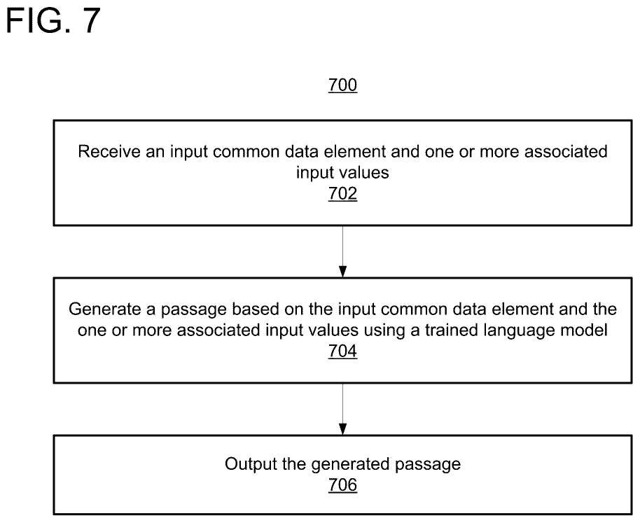

At step 110 of FIG. 1, the trained language model is output. For example, the trained language model can be output by storing the trained language model on a memory or storage of a computer system (e.g., memory 1110 or storage 1112 of computer 1102 of FIG. 11) or by transmitting the trained language model to a remote computer system (e.g., computer 1102 of FIG. 11). In one embodiment, the trained machine learning based text encoder network may be applied during an online or inference stage to generate a passage, e.g., according to method 700 of FIG. 7, described in detail below.

FIG. 5 shows a workflow 500 for training and applying a PLM for mapping passages to common data elements, in accordance with one or more embodiments. The trained PLM may be applied during an online or inference stage, e.g., to perform steps 104 and 106 of FIG. 1.

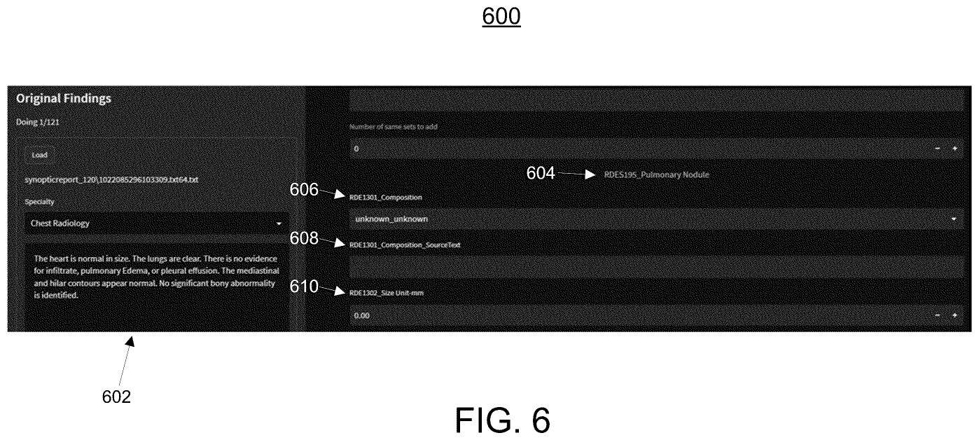

As shown in workflow 500, radiology domain-adapted PLM 502 comprises PLM sentence transformer 504 and PLM few shot classifier 506. PLM sentence transformer 504 and PLM few shot classifier 506 are trained with training dataset 508 radiology reports annotated with CDEs. In one embodiment, the radiology reports may be annotated with CDEs using an annotation tool, as shown in FIG. 6 described in detail below.

During inference/testing, the unseen radiology reports 522 are preprocessed to extract their sentences 524 which are classified by PLM few shot classifier 506 to select the RadElement sets (RDES) 510. The possible RadElement attributes 516 are the concepts in RadElements that are part of the selected RDES 510. PLM sentence transformer 504 is used to generate embeddings 512 for possible RadElement attributes 516 using their respective definitions in the RadElement ontology. These embeddings 512 are compared to the embeddings of the sentence 524 and the RadElement attributes 518 with the highest similarity are selected. Each RadElement attribute definition is comprised of possible attribute values, which gives us the possible set of RadElement values 520 for the sentence. PLM sentence transformer 504 is used to generate embeddings 514 for these possible RadElement values 520 and the embeddings of the sentence 524 are compared to these embeddings to give the RadElement value with the highest match. The chosen RadElement attributes 518 and their corresponding predicted values form the final output 526 of this embodiment in the inference framework.

FIG. 6 shows an interface 600 of an annotation tool for annotating radiological data with common data elements and associated values, in accordance with one or more embodiments. A user may interact with interface 600 to annotate passage 602 with common data element 604 defining a pulmonary nodule and value 606 defining composition, value 608 defining composition source text, and value 610 defining size. In one embodiment, a singe passage may correspond to multiple common data elements and thus the same passage 602 may be annotated with multiple common data elements. Similarly, multiple passages may be annotated with a single common data element.

FIG. 7 shows a method 700 for generating radiological passages using a language model, in accordance with one or more embodiments. The steps and sub-steps of method 700 may be performed by one or more suitable computing devices, such as, e.g., computer 1102 of FIG. 11.

At step 702 of FIG. 7, an input common data element and one or more associated input values are received. The input common data element and the one or more associated input values may be received via one or more prompts. A prompt refers to input to a language model and may comprise also include instructions for performing the medical analysis task as well as additional information. One example of a prompt comprising the input common data element and the one or more associated input values is as follows: “Generate a findings sentence for the following CDE and its value: 1. CDE: Pneumothorax Presence, Value: present; 2. CDE: Pneumothorax Lung Tissue Collapse, Value: complete; 3. CDE: Pneumothorax Side, Value: left; 4. CDE: Pneumothorax Size, Value: large.” The input common data element and the one or more associated input values may be received via a prompt, for example, from a computer system via one or more APIs (application programming interfaces) or from a user interacting with a computer system (e.g., computer 802 of FIG. 8).

At step 704 of FIG. 7, a radiology passage is generated based on the input common data element and the one or more associated input values using a trained language model. The trained language model is trained, for example, according to method 100 of FIG. 1 or workflow 200 of FIG. 2. The trained language model may be, for example, an LLM or any other suitable language model. The trained language model receives the input common data element and the one or more associated input values via one or more prompts and generates as output the radiology passage. The radiology passage may include, for example, a phrase, a sentence, a paragraph, a section, or any other portion related to medical imaging. Continuing the example of the prompt received at step 702 of FIG. 7, the generate passage may be: “Large left tension pneumothorax with complete collapse of the left lung is observed.”

At step 706 of FIG. 7, the generated passage is output. For example, the generated passage can be output by displaying the generated passage on a display device of a computer system (e.g., I/O 1108 of computer 1102 of FIG. 11), storing the generated passage on a memory or storage of a computer system (e.g., memory 1110 or storage 1112 of computer 1102 of FIG. 11), or by transmitting the generated passage to a remote computer system (e.g., computer 1102 of FIG. 11). In one embodiment, the generated passage is output to a document for generating a radiology report.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based encoder network utilized at step 104, the machine learning based classifier model utilized at step 106, and the language model utilized at step 108 of FIG. 1, Ner 208, entity linking 212, language model 224, and final model 228 of FIG. 2, sapBERT 304-A, MedLinker 304-B, BM25-based linker 304-C, and text similarity calculator 306 of FIG. 3, radiology domain-adapted PLM 502, PLM sentence transformer 504, and PLM few shot classifier 506 of FIG. 5, and the trained language model utilized at step 704 of FIG. 7, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

FIG. 8 shows an embodiment of an artificial neural network 800 that may be used to implement one or more machine learning models described herein. Alternative terms for “artificial neural network” are “neural network”, “artificial neural net” or “neural net”.

The artificial neural network 800 comprises nodes 820, . . . , 832 and edges 840, . . . 842, wherein each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832, it is also possible that the first node 820, . . . , 832 and the second node 820, . . . , 832 are identical. For example, in FIG. 8 the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as “ingoing edge” for the second node 820, . . . , 832 and as “outgoing edge” for the first node 820, . . . , 832.

In this embodiment, the nodes 820, . . . , 832 of the artificial neural network 800 can be arranged in layers 810, . . . , 813, wherein the layers can comprise an intrinsic order introduced by the edges 840, . . . , 842 between the nodes 820, . . . , 832. In particular, edges 840, . . . , 842 can exist only between neighboring layers of nodes. In the displayed embodiment, there is an input layer 810 comprising only nodes 820, . . . , 822 without an incoming edge, an output layer 813 comprising only nodes 831, 832 without outgoing edges, and hidden layers 811, 812 in-between the input layer 810 and the output layer 813. In general, the number of hidden layers 811, 812 can be chosen arbitrarily. The number of nodes 820, . . . , 822 within the input layer 810 usually relates to the number of input values of the neural network, and the number of nodes 831, 832 within the output layer 813 usually relates to the number of output values of the neural network.

In particular, a (real) number can be assigned as a value to every node 820, . . . , 832 of the neural network 800. Here, x⁽ⁿ⁾ᵢ denotes the value of the i-th node 820, . . . 832 of the n-th layer 810, . . . , 813. The values of the nodes 820, . . . , 822 of the input layer 810 are equivalent to the input values of the neural network 800, the values of the nodes 831, 832 of the output layer 813 are equivalent to the output value of the neural network 800. Furthermore, each edge 840, . . . , 842 can comprise a weight being a real number, in particular, the weight is a real number within the interval [−1, 1] or within the interval [0, 1]. Here, w^{(m,n)}_{i,j} denotes the weight of the edge between the i-th node 820, . . . , 832 of the m-th layer 810, . . . , 813 and the j-th node 820, . . . , 832 of the n-th layer 810, . . . , 813. Furthermore, the abbreviation w (n); j is defined for the weight w^{(n,n+1)}_{i,j}.

In particular, to calculate the output values of the neural network 800, the input values are propagated through the neural network. In particular, the values of the nodes 820, . . . , 832 of the (n+1)-th layer 810, . . . , 813 can be calculated based on the values of the nodes 820, . . . , 832 of the n-th layer 810, . . . , 813 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 810 are given by the input of the neural network 800, wherein values of the first hid-den layer 811 can be calculated based on the values of the input layer 810 of the neural network, wherein values of the second hidden layer 812 can be calculated based in the values of the first hidden layer 811, etc.

In order to set the values w^{(m,n)}_{i,j} for the edges, the neural network 800 has to be trained using training data. In particular, training data comprises training input data and training output data (denoted as tᵢ). For a training step, the neural network 800 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 800 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 813, wherein f′ is the first derivative of the activation function, and t⁽ⁿ⁺¹⁾ⱼ is the comparison training value for the j-th node of the output layer 813.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

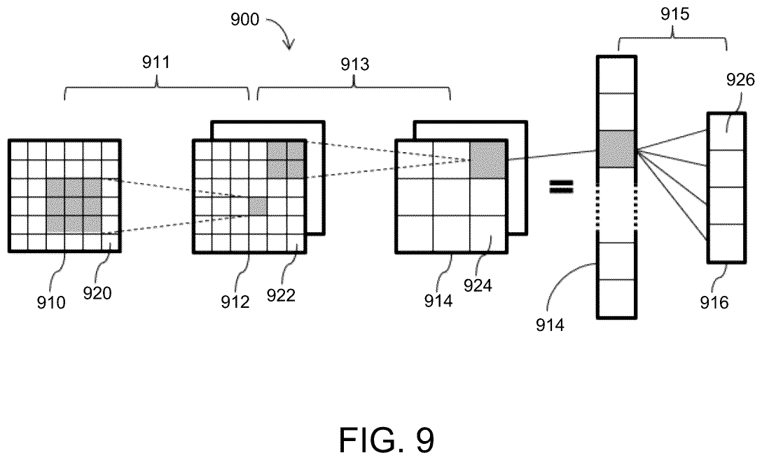

FIG. 9 shows an embodiment of a convolutional neural network 900 that may be used to implement one or more machine learning models described herein. In the displayed embodiment, the convolutional neural network comprises 900 an input node layer 910, a convolutional layer 911, a pooling layer 913, a fully connected layer 914 and an output node layer 916, as well as hidden node layers 912, 914. Alternatively, the convolutional neural network 900 can comprise several convolutional layers 911, several pooling layers 913 and several fully connected layers 915, as well as other types of layers. The order of the layers can be chosen arbitrarily, usually fully connected layers 915 are used as the last layers before the output layer 916.

In particular, within a convolutional neural network 900 nodes 920, 922, 924 of a node layer 910, 912, 914 can be considered to be arranged as a d-dimensional matrix or as a d-dimensional image. In particular, in the two-dimensional case the value of the node 920, 922, 924 indexed with i and j in the n-th node layer 910, 912, 914 can be denoted as x(n)[i,j]. However, the arrangement of the nodes 920, 922, 924 of one node layer 910, 912, 914 does not have an effect on the calculations executed within the convolutional neural network 900 as such, since these are given solely by the structure and the weights of the edges.

A convolutional layer 911 is a connection layer between an anterior node layer 910 (with node values x(n−1)) and a posterior node layer 912 (with node values x(n)). In particular, a convolutional layer 911 is characterized by the structure and the weights of the incoming edges forming a convolution operation based on a certain number of kernels. In particular, the structure and the weights of the edges of the convolutional layer 911 are chosen such that the values x(n) of the nodes 922 of the posterior node layer 912 are calculated as a convolution x(n)=K*x(n−1) based on the values x(n−1) of the nodes 920 anterior node layer 910, where the convolution * is defined in the two-dimensional case as

$${x}_{k}^{( n )} [ i , j ] = \begin{matrix}( K & {}^{*} {x}^{( n - 1 )} )\end{matrix} [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix(in this embodiment, a two-dimensional matrix), which is usually small compared to the number of nodes 920, 922 (e.g., a 3×3 matrix, or a 5×5 matrix). In particular, this implies that the weights of the edges in the convolution layer 911 are not independent, but chosen such that they produce said convolution equation. In particular, for a kernel being a 3×3 matrix, there are only 9 independent weights (each entry of the kernel matrix corresponding to one independent weight), irrespectively of the number of nodes 920, 922 in the anterior node layer 910 and the posterior node layer 912.

In general, convolutional neural networks 900 use node layers 910, 912, 914 with a plurality of channels, in particular, due to the use of a plurality of kernels in convolutional layers 911. In those cases, the node layers can be considered as (d+1)-dimensional matrices (the first dimension indexing the channels). The action of a convolutional layer 911 is then a two-dimensional example defined as

$${x}^{{( n )}_{b}} [ i , j ] = {\sum }_{a} {K}_{a , b} {}^{*} {x}^{{( n - 1 )}_{a}} [ i , j ] = {\sum }_{a} {\sum }_{{i}^{′}} {\sum }_{{j}^{′}} {K}_{a , b} [ i - {i}^{′} , j - {j}^{′} ]$$

where x⁽ⁿ⁻¹⁾^{ₐ} corresponds to the a-th channel of the anterior node layer 910, x⁽ⁿ⁾^{_{b}} corresponds to the b-th channel of the posterior node layer 912 and K_{a,b} corresponds to one of the kernels. If a convolutional layer 911 acts on an anterior node layer 910 with A channels and outputs a posterior node layer 912 with B channels, there are A·B independent d-dimensional kernels K_{a,b}.

In general, in convolutional neural networks 900 activation functions are used. In this embodiment re ReLU (acronym for “Rectified Linear Units”) is used, with R (z)=max(0, z), so that the action of the convolutional layer 911 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tan h or Softmax.

In the displayed embodiment, the input layer 910 comprises 36 nodes 920, arranged as a two-dimensional 6×6 matrix. The first hidden node layer 912 comprises 72 nodes 922, arranged as two two-dimensional 6×6 matrices, each of the two matrices being the result of a convolution of the values of the input layer with a 3×3 kernel within the convolutional layer 911. Equivalently, the nodes 922 of the first hidden node layer 912 can be interpreted as arranged as a three-dimensional 2×6×6 matrix, wherein the first dimension correspond to the channel dimension.

The advantage of using convolutional layers 911 is that spatially local correlation of the input data can exploited by enforcing a local connectivity pattern between nodes of adjacent layers, in particular by each node being connected to only a small region of the nodes of the preceding layer.

A pooling layer 913 is a connection layer between an anterior node layer 912 (with node values x(n−1)) and a posterior node layer 914 (with node values x(n)). In particular, a pooling layer 913 can be characterized by the structure and the weights of the edges and the activation function forming a pooling operation based on a non-linear pooling function f. For example, in the two-dimensional case the values x(n) of the nodes 924 of the posterior node layer 914 can be calculated based on the values x(n−1) of the nodes 922 of the anterior node layer 912 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 913 the number of nodes 922, 924 can be reduced, by re-placing a number d1·d2 of neighboring nodes 922 in the anterior node layer 912 with a single node 922 in the posterior node layer 914 being calculated as a function of the values of said number of neighboring nodes. In particular, the pooling function f can be the max-function, the average or the L2-Norm. In particular, for a pooling layer 913 the weights of the incoming edges are fixed and are not modified by training.

The advantage of using a pooling layer 913 is that the number of nodes 922, 924 and the number of parameters is reduced. This leads to the amount of computation in the network being reduced and to a control of overfitting.

In the displayed embodiment, the pooling layer 913 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 900 are fully connected layers 915. A fully connected layer 915 is a connection layer between an anterior node layer 914 and a posterior node layer 916. A fully connected layer 913 can be characterized by the fact that a majority, in particular, all edges between nodes 914 of the anterior node layer 914 and the nodes 916 of the posterior node layer are present, and wherein the weight of each of these edges can be adjusted individually.

In this embodiment, the nodes 924 of the anterior node layer 914 of the fully connected layer 915 are displayed both as two-dimensional matrices, and additionally as non-related nodes (indicated as a line of nodes, wherein the number of nodes was reduced for a better presentability). This operation is also denoted as “flattening”. In this embodiment, the number of nodes 926 in the posterior node layer 916 of the fully connected layer 915 smaller than the number of nodes 924 in the anterior node layer 914. Alternatively, the number of nodes 926 can be equal or larger.

Furthermore, in this embodiment the Softmax activation function is used within the fully connected layer 915. By applying the Softmax function, the sum the values of all nodes 926 of the output layer 916 is 1, and all values of all nodes 926 of the output layer 916 are real numbers between 0 and 1. In particular, if using the convolutional neural network 900 for categorizing input data, the values of the output layer 916 can be interpreted as the probability of the input data falling into one of the different categories.

In particular, convolutional neural networks 900 can be trained based on the backpropagation algorithm. For preventing overfitting, methods of regularization can be used, e.g., dropout of nodes 920, . . . , 924, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

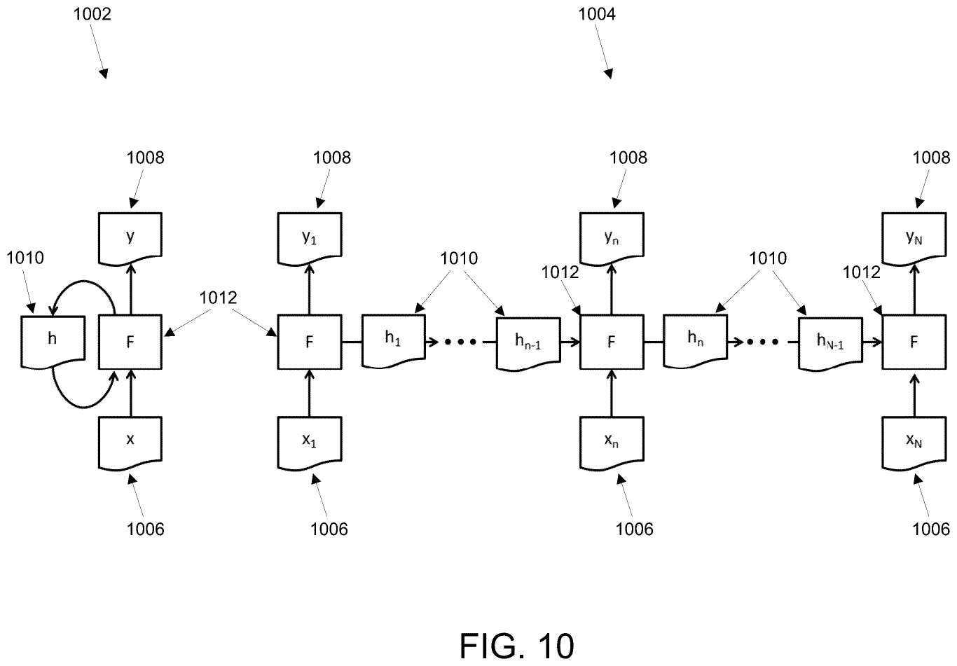

FIG. 10 shows the schematic structure of a recurrent machine learning model F, both in a recurrent representation 1002 and in an unfolded representation 1004, that may be used to implement one or more machine learning models described herein. The recurrent machine learning model takes as input several input datasets x, x₁, . . . , x_{N} 1006 and creates a corresponding set of output datasets y, y₁, . . . , y_{N} 1008. Furthermore, the output depends on a so-called hidden vector h, h₁, . . . , h_{N} 1010, which implicitly comprises information about input datasets previously used as input for the recurrent machine learning model F 1012. By using these hidden vectors h, h₁, . . . , h_{N} 1010, a sequentiality of the input datasets can be leveraged.

In a single step of the processing, the recurrent machine learning model F 1012 takes as input the hidden vector hₙ₋₁ created within the previous step and an input dataset Xn. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 1012 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 1012 that were trained based on training datasets before do not change between the different processing steps.

In particular, the output data and the hidden vector of a processing step depend on all the previous input datasets used in the previous steps. yₙ=F(y) (Xn, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1-3, 5, or 7. Certain steps or functions of the methods and workflows described herein, including one or more of the steps or functions of FIG. 1-3, 5, or 7, may be performed by a server or by another processor in a network-based cloud-computing system. Certain steps or functions of the methods and workflows described herein, including one or more of the steps of FIG. 1-3, 5, or 7, may be performed by a client computer in a network-based cloud computing system. The steps or functions of the methods and workflows described herein, including one or more of the steps of FIG. 1-3, 5, or 7, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1-3, 5, or 7, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1102 that may be used to implement systems, apparatuses, and methods described herein is depicted in FIG. 11. Computer 1102 includes a processor 1104 operatively coupled to a data storage device 1112 and a memory 1110. Processor 1104 controls the overall operation of computer 1102 by executing computer program instructions that define such operations. The computer program instructions may be stored in data storage device 1112, or other computer readable medium, and loaded into memory 1110 when execution of the computer program instructions is desired. Thus, the method and workflow steps or functions of FIG. 1-3, 5, or 7 can be defined by the computer program instructions stored in memory 1110 and/or data storage device 1112 and controlled by processor 1104 executing the computer program instructions. For example, the computer program instructions can be implemented as computer executable code programmed by one skilled in the art to perform the method and workflow steps or functions of FIG. 1-3, 5, or 7. Accordingly, by executing the computer program instructions, the processor 1104 executes the method and workflow steps or functions of FIG. 1-3, 5, or 7. Computer 1102 may also include one or more network interfaces 1106 for communicating with other devices via a network. Computer 1102 may also include one or more input/output devices 1108 that enable user interaction with computer 1102 (e.g., display, keyboard, mouse, speakers, buttons, etc.).

Processor 1104 may include both general and special purpose microprocessors, and may be the sole processor or one of multiple processors of computer 1102. Processor 1104 may include one or more central processing units (CPUs), for example. Processor 1104, data storage device 1112, and/or memory 1110 may include, be supplemented by, or incorporated in, one or more application-specific integrated circuits (ASICs) and/or one or more field programmable gate arrays (FPGAs).

Data storage device 1112 and memory 1110 each include a tangible non-transitory computer readable storage medium. Data storage device 1112, and memory 1110, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1108 may include peripherals, such as a printer, scanner, display screen, etc. For example, input/output devices 1108 may include a display device such as a cathode ray tube (CRT) or liquid crystal display (LCD) monitor for displaying information to the user, a keyboard, and a pointing device such as a mouse or a trackball by which the user can provide input to computer 1102.

An image acquisition device 1114 can be connected to the computer 1102 to input image data (e.g., medical images) to the computer 1102. It is possible to implement the image acquisition device 1114 and the computer 1102 as one device. It is also possible that the image acquisition device 1114 and the computer 1102 communicate wirelessly through a network. In a possible embodiment, the computer 1102 can be located remotely with respect to the image acquisition device 1114.

Any or all of the systems, apparatuses, and methods discussed herein may be implemented using one or more computers such as computer 1102.

One skilled in the art will recognize that an implementation of an actual computer or computer system may have other structures and may contain other components as well, and that FIG. 11 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving an input common data element and one or more associated input values; generating a radiology passage based on the input common data element and the one or more associated input values using a trained language model; and outputting the generated radiology passage.

Illustrative embodiment 2. The computer-implemented method of illustrative embodiment 1, wherein the trained language model is trained by: receiving text-based radiological data comprising one or more passages; extracting a concept from each of the one or more passages; for each respective passage of the one or more passages, mapping the concept extracted from the respective passage to a common data element and one or more associated values, thereby resulting in pairs of 1) the respective passage and 2) the common data element and the one or more associated values; and training the language model for generating the radiology passage based on the pairs of 1) the passages and 2) the common data elements and the one or more associated values.

Illustrative embodiment 3. The computer-implemented method of illustrative embodiment 2, wherein training the language model for generating the radiology passage based on the pairs of 1) the passages and 2) the common data elements and the one or more associated values comprises: fine-tuning the language model via instruction tuning based on the pairs of 1) the passages and 2) the common data elements and the one or more associated values comprises.

Illustrative embodiment 4. The computer-implemented method of any one of illustrative embodiments 2-3, wherein training the language model for generating the radiology passage based on the pairs of 1) the passages and 2) the common data elements and the one or more associated values comprises: training the language model via reinforcement learning by evaluating the common data elements against passaged generated by the language model.

Illustrative embodiment 5. The computer-implemented method of any one of illustrative embodiments 2-4, wherein mapping the concept extracted from the respective passage to a common data element and one or more associated values comprises: in response to determining that the common data element is not available for mapping, mapping the concept extracted from the respective passage to an entity of an ontology.

Illustrative embodiment 6. The computer-implemented method of any one of illustrative embodiments 2-5, wherein mapping the concept extracted from the respective passage to an entity of an ontology comprises: determining a ranking of entities for the ontology from each of a plurality of entity linking models based on the concept extracted from the respective passage; and determining the entity of the ontology based on the ranking of the entities.

Illustrative embodiment 7. The computer-implemented method of any one of illustrative embodiments 2-6, wherein: extracting a concept from each of the one or more passages comprises encoding each of the one or more passages into features using a machine learning based encoder network; and mapping the concept extracted from the respective passage to a common data element and one or more associated values comprises classifying the concept to a common data element and the one or more associated values using a machine learning based classifier model.

Illustrative embodiment 8. An apparatus comprising: means for receiving an input common data element and one or more associated input values; means for generating a radiology passage based on the input common data element and the one or more associated input values using a trained language model; and means for outputting the generated radiology passage.

Illustrative embodiment 9. The apparatus of illustrative embodiment 8, wherein the trained language model is trained by: receiving text-based radiological data comprising one or more passages; extracting a concept from each of the one or more passages; for each respective passage of the one or more passages, mapping the concept extracted from the respective passage to a common data element and one or more associated values, thereby resulting in pairs of 1) the respective passage and 2) the common data element and the one or more associated values; and training the language model for generating the radiology passage based on the pairs of 1) the passages and 2) the common data elements and the one or more associated values.

Illustrative embodiment 10. The apparatus of illustrative embodiment 9, wherein training the language model for generating the radiology passage based on the pairs of 1) the passages and 2) the common data elements and the one or more associated values comprises: fine-tuning the language model via instruction tuning based on the pairs of 1) the passages and 2) the common data elements and the one or more associated values comprises.

Illustrative embodiment 11. The apparatus of any one of illustrative embodiments 9-10, wherein training the language model for generating the radiology passage based on the pairs of 1) the passages and 2) the common data elements and the one or more associated values comprises: training the language model via reinforcement learning by evaluating the common data elements against passaged generated by the language model.

Illustrative embodiment 12. The apparatus of any one of illustrative embodiments 9-11, wherein mapping the concept extracted from the respective passage to a common data element and one or more associated values comprises: in response to determining that the common data element is not available for mapping, mapping the concept extracted from the respective passage to an entity of an ontology.

Illustrative embodiment 13. The apparatus of any one of illustrative embodiments 9-12, wherein mapping the concept extracted from the respective passage to an entity of an ontology comprises: determining a ranking of entities for the ontology from each of a plurality of entity linking models based on the concept extracted from the respective passage; and determining the entity of the ontology based on the ranking of the entities.

Illustrative embodiment 14. A non-transitory computer-readable storage medium comprising instructions which, when executed by a computer, cause the computer to carry out operations comprising: receiving an input common data element and one or more associated input values; generating a radiology passage based on the input common data element and the one or more associated input values using a trained language model; and outputting the generated radiology passage.

Illustrative embodiment 15. The non-transitory computer-readable storage medium of illustrative embodiment 14, wherein the trained language model is trained by: receiving text-based radiological data comprising one or more passages; extracting a concept from each of the one or more passages; for each respective passage of the one or more passages, mapping the concept extracted from the respective passage to a common data element and one or more associated values, thereby resulting in pairs of 1) the respective passage and 2) the common data element and the one or more associated values; and training the language model for generating the radiology passage based on the pairs of 1) the passages and 2) the common data elements and the one or more associated values.

Illustrative embodiment 16. The non-transitory computer-readable storage medium of illustrative embodiment 15, wherein training the language model for generating the radiology passage based on the pairs of 1) the passages and 2) the common data elements and the one or more associated values comprises: fine-tuning the language model via instruction tuning based on the pairs of 1) the passages and 2) the common data elements and the one or more associated values comprises.

Illustrative embodiment 17. The non-transitory computer-readable storage medium of any one of illustrative embodiments 15-16, wherein training the language model for generating the radiology passage based on the pairs of 1) the passages and 2) the common data elements and the one or more associated values comprises: training the language model via reinforcement learning by evaluating the common data elements against passaged generated by the language model.

Illustrative embodiment 18. The non-transitory computer-readable storage medium of any one of illustrative embodiments 15-17, wherein: extracting a concept from each of the one or more passages comprises encoding each of the one or more passages into features using a machine learning based encoder network; and mapping the concept extracted from the respective passage to a common data element and one or more associated values comprises classifying the concept to a common data element and the one or more associated values using a machine learning based classifier model.

Illustrative embodiment 19. A computer-implemented method comprising: receiving text-based radiological data comprising one or more passages; extracting a concept from each of the one or more passages; for each respective passage of the one or more passages, mapping the concept extracted from the respective passage to a common data element and one or more associated values, thereby resulting in pairs of 1) the respective passage and 2) the common data element and the one or more associated values; training a language model for generating a radiology passage based on the pairs of 1) the passages and 2) the common data elements and the one or more associated values; and outputting the trained language model.

Illustrative embodiment 20. The computer-implemented method of illustrative embodiment 19, wherein training the language model for generating the radiology passage based on the pairs of 1) the passages and 2) the common data elements and the one or more associated values comprises: fine-tuning the language model via instruction tuning based on the pairs of 1) the passages and 2) the common data elements and the one or more associated values comprises.

Illustrative embodiment 21. The computer-implemented method of any one of illustrative embodiments 19-20, wherein training the language model for generating the radiology passage based on the pairs of 1) the passages and 2) the common data elements and the one or more associated values comprises: training the language model via reinforcement learning by evaluating the common data elements against passaged generated by the language model.

Illustrative embodiment 22. The computer-implemented method of any one of illustrative embodiments 19-21, further comprising: receiving an input common data element and one or more associated input values; generating a radiology passage based on the input common data element and the one or more associated input values using the trained language model; and outputting the generated radiology passage.

1. A computer-implemented method comprising:
receiving an input common data element and one or more associated input values;
generating a radiology passage based on the input common data element and the one or more associated input values using a trained language model; and
outputting the generated radiology passage.

2. The computer-implemented method of claim 1, wherein the trained language model is trained by:
receiving text-based radiological data comprising one or more passages;
extracting a concept from each of the one or more passages;
for each respective passage of the one or more passages, mapping the concept extracted from the respective passage to a common data element and one or more associated values, thereby resulting in pairs of 1) the respective passage and 2) the common data element and the one or more associated values; and
training the language model for generating the radiology passage based on the pairs of 1) the passages and 2) the common data elements and the one or more associated values.

3. The computer-implemented method of claim 2, wherein training the language model for generating the radiology passage based on the pairs of 1) the passages and 2) the common data elements and the one or more associated values comprises:
fine-tuning the language model via instruction tuning based on the pairs of 1) the passages and 2) the common data elements and the one or more associated values comprises.

4. The computer-implemented method of claim 2, wherein training the language model for generating the radiology passage based on the pairs of 1) the passages and 2) the common data elements and the one or more associated values comprises:
training the language model via reinforcement learning by evaluating the common data elements against passaged generated by the language model.

5. The computer-implemented method of claim 2, wherein mapping the concept extracted from the respective passage to a common data element and one or more associated values comprises:
in response to determining that the common data element is not available for mapping, mapping the concept extracted from the respective passage to an entity of an ontology.

6. The computer-implemented method of claim 5, wherein mapping the concept extracted from the respective passage to an entity of an ontology comprises:
determining a ranking of entities for the ontology from each of a plurality of entity linking models based on the concept extracted from the respective passage; and
determining the entity of the ontology based on the ranking of the entities.

7. The computer-implemented method of claim 2, wherein:
extracting a concept from each of the one or more passages comprises encoding each of the one or more passages into features using a machine learning based encoder network; and
mapping the concept extracted from the respective passage to a common data element and one or more associated values comprises classifying the concept to a common data element and the one or more associated values using a machine learning based classifier model.

8. An apparatus comprising:
means for receiving an input common data element and one or more associated input values;
means for generating a radiology passage based on the input common data element and the one or more associated input values using a trained language model; and
means for outputting the generated radiology passage.

9. The apparatus of claim 8, wherein the trained language model is trained by:
receiving text-based radiological data comprising one or more passages;
extracting a concept from each of the one or more passages;
for each respective passage of the one or more passages, mapping the concept extracted from the respective passage to a common data element and one or more associated values, thereby resulting in pairs of 1) the respective passage and 2) the common data element and the one or more associated values; and
training the language model for generating the radiology passage based on the pairs of 1) the passages and 2) the common data elements and the one or more associated values.

10. The apparatus of claim 9, wherein training the language model for generating the radiology passage based on the pairs of 1) the passages and 2) the common data elements and the one or more associated values comprises:
fine-tuning the language model via instruction tuning based on the pairs of 1) the passages and 2) the common data elements and the one or more associated values comprises.

11. The apparatus of claim 9, wherein training the language model for generating the radiology passage based on the pairs of 1) the passages and 2) the common data elements and the one or more associated values comprises:
training the language model via reinforcement learning by evaluating the common data elements against passaged generated by the language model.

12. The apparatus of claim 9, wherein mapping the concept extracted from the respective passage to a common data element and one or more associated values comprises:
in response to determining that the common data element is not available for mapping, mapping the concept extracted from the respective passage to an entity of an ontology.

13. The apparatus of claim 12, wherein mapping the concept extracted from the respective passage to an entity of an ontology comprises:
determining a ranking of entities for the ontology from each of a plurality of entity linking models based on the concept extracted from the respective passage; and
determining the entity of the ontology based on the ranking of the entities.

14. A non-transitory computer-readable storage medium comprising instructions which, when executed by a computer, cause the computer to carry out operations comprising:
receiving an input common data element and one or more associated input values;
generating a radiology passage based on the input common data element and the one or more associated input values using a trained language model; and
outputting the generated radiology passage.

15. The non-transitory computer-readable storage medium of claim 14, wherein the trained language model is trained by:
receiving text-based radiological data comprising one or more passages;
extracting a concept from each of the one or more passages;
for each respective passage of the one or more passages, mapping the concept extracted from the respective passage to a common data element and one or more associated values, thereby resulting in pairs of 1) the respective passage and 2) the common data element and the one or more associated values; and
training the language model for generating the radiology passage based on the pairs of 1) the passages and 2) the common data elements and the one or more associated values.

16. The non-transitory computer-readable storage medium of claim 15, wherein training the language model for generating the radiology passage based on the pairs of 1) the passages and 2) the common data elements and the one or more associated values comprises:
fine-tuning the language model via instruction tuning based on the pairs of 1) the passages and 2) the common data elements and the one or more associated values comprises.

17. The non-transitory computer-readable storage medium of claim 15, wherein training the language model for generating the radiology passage based on the pairs of 1) the passages and 2) the common data elements and the one or more associated values comprises:
training the language model via reinforcement learning by evaluating the common data elements against passaged generated by the language model.

18. The non-transitory computer-readable storage medium of claim 15, wherein:
extracting a concept from each of the one or more passages comprises encoding each of the one or more passages into features using a machine learning based encoder network; and
mapping the concept extracted from the respective passage to a common data element and one or more associated values comprises classifying the concept to a common data element and the one or more associated values using a machine learning based classifier model.

19. A computer-implemented method comprising:
receiving text-based radiological data comprising one or more passages;
extracting a concept from each of the one or more passages;
for each respective passage of the one or more passages, mapping the concept extracted from the respective passage to a common data element and one or more associated values, thereby resulting in pairs of 1) the respective passage and 2) the common data element and the one or more associated values;
training a language model for generating a radiology passage based on the pairs of 1) the passages and 2) the common data elements and the one or more associated values; and
outputting the trained language model.

20. The computer-implemented method of claim 19, wherein training the language model for generating the radiology passage based on the pairs of 1) the passages and 2) the common data elements and the one or more associated values comprises:
fine-tuning the language model via instruction tuning based on the pairs of 1) the passages and 2) the common data elements and the one or more associated values comprises.

21. The computer-implemented method of claim 19, wherein training the language model for generating the radiology passage based on the pairs of 1) the passages and 2) the common data elements and the one or more associated values comprises:
training the language model via reinforcement learning by evaluating the common data elements against passaged generated by the language model.

22. The computer-implemented method of claim 19, further comprising:
receiving an input common data element and one or more associated input values;
generating a radiology passage based on the input common data element and the one or more associated input values using the trained language model; and
outputting the generated radiology passage.
